(19) United States
(12) Patent Application Publication
(10) Pub. No.: US 2005/0208592 A1
(43) Pub. Date: Sep. 22, 2005

(54) Method and apparatus for the detection of microorganisms

(75) Inventors: Eric Caron, Ste-Therese (CA); John H. T. Luong, Montreal (CA); Keith B. Male, Montreal (CA); Rosemonde Mandeville, Ste-Therese (CA); Alberto Mazza, Rosemere (CA)

Correspondence Address: OGILVY RENAULT LLP, 1981 MCGILL COLLEGE AVENUE, SUITE 1600, MONTREAL, QC, H3A2Y3, CA

(73) Assignee: National Research Council of Canada

(21) Appl. No.: 11/081,687

(22) Filed: Mar. 17, 2005

Related U.S. Application Data
(60) Provisional application No. 60/553,544, filed on Mar. 17, 2004.

(51) Int. Cl. G01N 33/53; A61N 1/18

(52) U.S. Cl. 435/7.1

(57) ABSTRACT

The present application discloses a method for detecting a viable microorganism in a sample. The method may comprise (i) providing a detecting electrode and a counter electrode, (ii) contacting said sample with said detecting electrode and said counter electrode and (iii) measuring a difference in impedance between said detecting electrode and said counter electrode. The detecting electrode may comprise gold nanoparticles deposited thereon and/or a capture molecule. The capture molecule may be able to bind to the microorganism. A redox mediator can also be added to the sample prior to the impedance measurement. Also disclosed are related apparatuses and uses.

### CROSS-REFERENCE TO RELATED APPLICATIONS

This application claims priority on U.S. application 60/553,544, filed Mar. 17, 2004, the entire content of which is hereby incorporated by reference.

### TECHNICAL FIELD

This invention relates to apparatus, methods and related uses for detecting a microorganism in a sample. The invention also relates to the use of a detecting electrode comprising gold nanoparticles and/or capture molecules for the detection of a microorganism in a sample.

### BACKGROUND OF THE INVENTION

The detection of viruses and microorganisms such as bacteria and their spores are routinely monitored by bacterial culture methods, PCR or enzyme-linked immunoassay (ELISA) techniques. However, the ELISA technique is rather time consuming and/or costly due to the necessity of adding a labeling step to visualize the binding of the microorganism. Classical bacterial culture methods are time consuming and will give accurate/sensitive results within 2-7 days, whereas, without amplification step, PCR or ELISA techniques are faster but have a detection limits in the range of 10⁵-10⁶ cells/mL.

In contrast, impedance measurements by Electric Cell-Substrate Impedance Sensing (ECIS) have been extensively exploited previously for monitoring the behavior of eukaryotic cells, such as mammalian and insect cells. ECIS can be used to monitor attachment, motility, mortality and cytotoxicity of eukaryotic cells. The apparatus used usually includes a small gold electrode (250 μm diameter) deposited on the bottom of culture wells and immersed in a culture medium containing eukaryotic cells. The eukaryotic cells have a tendency to drift downwards and attach to the surface of the electrode. Using culture medium as the electrolyte, a constant current source applies a small AC current of ˜1 μA at 4,000-5,000 Hz between the small detecting electrode and a large counter electrode and the resulting voltage is monitored by a lock-in amplifier. The deposition and attachment of cells on the electrode act as insulating particles because their plasma membrane will interfere with the free space immediately above the electrode for current flow. Owing to its smaller size compared to the counter electrode, the detecting electrode will dominate the overall impedance in the circuit, which will increase in a few hours as cells gradually augment the surface they occupy on the gold surface. The size of the electrode will restrict the maximum cell concentration on the electrode surface to about 100-150 cells. The binding of the cells to the surface may be enhanced by the attachment of capture molecules to the gold surface. For example, both Concavalin A and fibronectin have been shown to enhance the impedance responses for insect and mammalian cells, respectively. The ECIS technique has never been used for the detection of smaller targets such as prokaryotic cells, viruses and prions. Further, it has never been used to discriminate between two very closely related targets.

It would be highly desirable to be provided with a rapid, sensible and reproducible method for detecting microorganisms in various samples and an apparatus for conducting such method. It would also be highly desirable to be provided with a more cost-effective method for detecting the microorganisms and an apparatus for conducting such method. Further, it would be highly desirable to be provided with a real-time, quantitative method for detecting microorganisms and apparatus for conducting such methods.

### SUMMARY OF THE INVENTION

The present inventions provides pparatus for detecting a viable microorganism, method for detecting a microorganism and related uses.

In a first aspect, the present invention provides an apparatus for detecting a viable microorganism in a sample, said apparatus comprising (i) a detecting electrode comprising gold nanoparticles deposited thereon, (ii) a counter electrode and (iii) a capture molecule. In an embodiment, the capture molecule is connectable to said detecting electrode and the capture molecule is able to bind to said microorganism. In another embodiment, the detecting electrode is a gold electrode. In another embodiment, the average size of the gold nanoparticles is from about 15 nm to about 300 nm. In still another embodiment, the diameter of the detecting electrode is of about 100 μm to about 250 μm. In yet another embodiment, the apparatus further comprises a first module for applying an electrical signal to the sample. In an embodiment, the first module is connectable to the detecting electrode and the counter electrode. In yet another embodiment, thee electrical signal has an alternating current from about 1 μA to about 3 μA. In another embodiment, the electrical signal has a potential of about 1.0 V to about 3.0 V and, in a further embodiment, a potential of about 1.5 V. In an embodiment, the characteristics of the signal between the detecting and counter electrodes can be measured by various means known to those skilled in the art. In an embodiment, the apparatus further comprises a second module for measuring a difference in voltage between the detecting electrode and the counter electrode. In an embodiment, the second module is connectable to the detecting electrode and the counter electrode. In yet another embodiment, the second module comprises an amplifier. In still another embodiment, the apparatus further comprises a third module for measuring a first impedance between the detecting electrode and the counter electrode. In an embodiment, the third module is connectable to the detecting electrode and the counter electrode. In a further embodiment, the apparatus further comprises a forth module for comparing the first impedance with a control impedance. In still a further embodiment, the forth module is connectable to said third module. In yet another embodiment, the control impedance is selected from the group consisting of an impedance of the sample measured at an earlier time, an impedance of a control sample substantially free of microorganism and a reference impedance. In yet another embodiment, the capture molecule is selected from the group consisting of an antibody, a phage, an amino acid and a protein. In an embodiment, the antibody is directed against Escherichia coli. In another embodiment, the phage is capable of binding to Escherichia coli. In still a further embodiment, the microorganism is selected from the group consisting of a bacterium, a fungus, a mold, a spore, a virus and a prion. In an embodiment, the microorganism is a bacterium. In another embodiment, the bacterium is Escherichia coli.

In another aspect, the present invention provides a method for detecting a viable microorganism, the method comprises a) providing a detecting electrode and a counter electrode, the detecting electrode comprising (i) gold nanoparticles deposited thereon and (ii) a capture molecule, the capture molecule being able to bind to the microorganism; b) contacting a media-comprising sample with the detecting electrode and the counter electrode; c) measuring a first impedance between the detecting electrode and the counter electrode; d) comparing the first impedance with a control impedance, the control impedance is selected from the group consisting of an impedance between the detecting electrode and the counter electrode of the sample at an earlier time, an impedance between the detecting electrode and the counter electrode in a sample substantially free of microorganism and a reference impedance; and wherein an increase of the first impedance with respect to the control impedance is indicative of the presence of said viable microorganism. In an embodiment, the detecting electrode, the gold nanoparticles used has been described above. In another embodiment, the method comprises applying an electrical signal to the media-comprising sample. In an embodiment, the electrical signal used has been described above. In another embodiment, the capture molecule is selected from the group consisting of an antibody, a phage, an amino acid and a protein. In an embodiment, the capture molecule used has been described above. In yet another embodiment, the microorganism is selected from the group consisting of a bacterium, a fungus, a mold, a spore, a virus and a prion. In an embodiment, the microorganism used has been described above. In yet another embodiment, the method comprises adding a redox mediator to the media-comprising sample. In yet another embodiment, the redox mediator is a potassium ferrocyanide/potassium ferricyanide solution.

In still another aspect, the present invention provides a method for detecting a viable microorganism, the method comprises a) providing a detecting electrode and a counter electrode; b) contacting a media-comprising sample containing a redox mediator with the detecting electrode and the counter electrode; c) measuring a first impedance between said detecting electrode and said counter electrode; d) comparing said first impedance with a control impedance, the control impedance being selected from the group consisting of an impedance between the detecting electrode and the counter electrode of the sample at an earlier time, an impedance between the detecting electrode and the counter electrode in a sample substantially free of microorganism and a reference difference in impedance; wherein an increase of the first impedance with respect to the control impedance is indicative of the presence of said viable microorganism.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the normalized resistance in function of time (hours) for electrode coated with phages specific to Escherichia coli (E. coli) K91 and submitted to various detecting applied potentials. For the measurements, the electrodes have been incubated in the presence of E. coli K91 for up to 10 hours.

FIG. 2 illustrates the normalized resistance in function of time (hours) for an electrode coated with phages specific to E. coli K91 and submitted to various frequencies. For the measurements, the electrodes have been incubated in the presence of E. coli K91 for up to 10 hours.

FIG. 3 illustrates the normalized resistance in function of time (hours) for an electrode coated with phages specific to E. coli K91 and incubated at various temperatures. For the measurements, the electrodes have been incubated in the presence of E. coli K91 for up to 10 hours.

FIG. 4 illustrates the normalized resistance in function of time (hours) for an electrode coated with phages specific to E. coli K91 and incubated with various inoculum volume. For the measurements, the electrodes have been incubated in the presence of E. coli K91 for up to 10 hours.

FIG. 5 illustrates the normalized resistance in function of time (hours) for a non-coated electrode (control), an electrode coated with an antibody specific to E. coli K91 (antibody) and an electrode coated with a biotinylated antibody specific to E. coli K91 (biotinylated antibody). For the measurements, the electrodes have been incubated in the presence of E. coli K91 for up to 25 hours.

FIG. 6 illustrates the normalized resistance in function of time (hours) for a non-coated electrode (control), an electrode coated with 1010 phages specific to E. coli K91 (1×10¹⁰), an electrode coated with 1011 phages specific to E. coli K91 (1×10¹¹) and an electrode coated with 10¹² phages specific to E. coli K91 (1×10¹²). For the measurements, the electrodes have been incubated in the presence of E. coli K91 for up to 25 hours.

FIG. 7 illustrates the normalized resistance in function of time (hours) for electrodes coated with phages specific to E. coli K91, either pre-incubated 2 h or 16 h with the phage solution. The electrodes have further been incubated in the presence of E. coli K91.

FIG. 8 illustrates the normalized resistance in function of time (hours) for electrodes coated with phages specific to E. coli K91 and incubated with fresh LB medium or 50% spent medium. The electrodes have been incubated in the presence of E. coli K91 for up to 10 hours.

FIG. 9 illustrates the normalized resistance in function of time (hours) for a non-coated electrode (Bt) and an electrode coated with phages specific to E. coli K91 (Phage+Bt). For the measurements, the electrodes have been incubated in the presence of Bacillus thuriengiensis (B. thuringiensis) for up to 16 hours.

FIG. 10 illustrates the normalized resistance in function of time (hours) for electrodes coated with phages specific to E. coli K91 and incubated with various concentrations of E. coli K91 (2×10⁵, 1×10⁶, 2×10⁶ or 1×10⁷ cells per electrode) for up to 12 hours.

FIG. 11 illustrates the normalized resistance in function of time (hours) for electrodes coated with (phage) or without (control) phages specific to E. coli K91 and having a diameter of 100 μm. The electrodes have been incubated in the presence of E. coli for up to 10 hours.

FIG. 12 shows atomic force images of electrodes and section analysis of unmodified electrodes (A) and electrodes modified by the deposition of gold nanoparticles (B).

FIG. 13 illustrates the normalized impedance in function of time (hours) for uncoated electrodes modified by the electrodeposition of gold nanoparticles at different deposition times. The electrodes have been incubated in the presence of E. coli for up to 10 hours.

FIG. 14 illustrates the normalized impedance in function of time (hours) for modified electrodes coated with phages (Modified electrode+phage), modified uncoated electrodes (Modified electrode+LB broth), modified electrode coated with casein (Modified electrode+0.5 mg/mL casein), modified electrode coated with cystein (Modified electrode+5 mg/mL cysteine) and unmodified electrode coated with phages (Control electrode+Phage).

FIG. 15 illustrates the normalized impedance in function of time (hours) for uncoated modified electrodes incubation with various concentration of E. coli K91. The electrodes have been incubated in the presence of E. coli for up to 30 hours.

FIG. 16 illustrates the normalized impedance in function of time (hours) for uncoated modified electrodes incubation with various concentration of B. thuringiensis. The electrodes have been further incubated in the presence of B. thuringiensis for up to 30 hours.

FIG. 17 illustrates the normalized impedance in function of time (hours) for modified and unmodified uncoated electrodes incubated in the presence or absence of the redox mediator. The electrodes have been incubated in the presence of E. coli K91 for up to 16 hours.

FIG. 18 illustrates the normalized impedance in function of time (hours) for electrodes treated with an initial solution of hydrogen tetrachloroaurate (III) trihydrate of varying concentration.

### DETAILED DESCRIPTION OF THE PREFERRED EMBODIMENT

In accordance with the present invention, there is provided methods for detecting a microorganism in a sample, as well as corresponding apparatus and uses.

In an embodiment, the present invention has an important advantage over other known techniques by those skilled in the art (such as ELISA or PCR) since it does not necessarily require the labeling of the reagents used and enables detection of cells and their behavior in real-time with quantitative results.

In an embodiment, the present invention can be used prior to known semi-quantitative/quantitative techniques for detecting microorganisms (such as PCR or ELISA) to augment the number of microorganisms to be detected, hence increasing the sensibility of the known techniques.

In another embodiment, the present invention has an important advantage over classical bacterial culture techniques since it enables detection of cells and their behavior more rapidly and in real-time with quantitative results.

In a first aspect, the present invention provides an apparatus for detecting a viable microorganism in a sample. In a first embodiment, the apparatus comprises a detecting electrode and a counter electrode.

As used herein, the term “detecting electrode” is defined as an electrode that dominates the overall impedance of the circuit. In an embodiment, the detecting electrode is much smaller in size than the counter electrode. The detecting electrode may be a gold electrode. The detecting electrode may also have a diameter of about 100 μm to about 250 μm. In another embodiment, the detecting electrode may be smaller in size than those used for the detection of eukaryotic cells. In fact, since bacterial cells (˜2×5-10 μm) are so much smaller compared to mammalian cells (25-50 μm), a higher concentration of bacteria are needed to cover the entire electrode surface. Therefore, a smaller gold electrode surface area may result in a quicker and higher impedance response for bacterial cells.

In another embodiment, the detecting electrode comprises gold nanoparticles deposited thereon. The gold nanoparticles may be deposited (e.g. electrodeposited) on the detecting electrode by the method described in the Examples below. The average size of the gold nanoparticles may be from about 15 to about 300 nm. In an embodiment, the average size of the gold nanoparticles may be from about 200 nm to about 300 nm, or from about 15 nm to about 40 nm. When a detecting electrode comprises gold nanoparticles, its surface may be modified (e.g. rougher in appearance) and its electrical properties may be modified. For example, such detecting electrodes may have a modulated mean roughness (e.g. a higher mean roughness, higher than 0.5 nm or 5 nm), a modulated impedance (e.g. a lower impedance, lower than 14,000 ω to about 2,000 ω), a modulated capacitance (e.g. a higher capacitance, higher than 3 nF, up to 25 nF) and/or a modulated resistance.

In a further embodiment, the apparatus also comprises a first module for applying a signal to the sample. The first module may, for example, be connectable to the detecting electrode and the counter electrode. As used herein, the term “connectable” refer to the ability of being connected. An object that is connectable may or may not be connected but is adapted to be connected. In an embodiment, the first module that is connected to the detecting electrode and the counter electrode. In an embodiment, the signal applied by the first module is an electrical signal. In another embodiment, the first module may also be able to apply an alternating current. In an embodiment, the signal may have an alternating current from about 1 μA to about 3 μA. In yet another embodiment, the signal may possess a potential of about 1.0 V to about 3.0 V, and in a further embodiment, a potential of about 1.5 V. The potential of the signal may be held constant during sampling or it may vary, according to the specific experimental conditions. In a further embodiment, the signal may have a frequency of about 1,000 Hz to about 4,000 Hz, and in a further embodiment, a frequency of about 4,000 Hz. The frequency of the signal may be held constant during sampling or it may vary, according to the specific experimental conditions. In yet a further embodiment, the first module may comprise a power source. The power source may, for example, be able to generate an alternating current.

In another aspect, the present invention provides an apparatus comprising a second module capable of measuring a difference in voltage between the two electrodes. The second module can, for example, be connectable to the detecting electrode and the counter electrode. In an embodiment, the second module is connected to the detecting electrode and the counter electrode. The second module may also, for example, measure the magnitude and phase of the voltage. The term “voltage” as used herein is defined as the numerical value of the electrical potential across or between any two points in an electric circuit. Volts are the unit of electromotive force or electric pressure. It is the electromotive force which, if steadily applied to a circuit having a resistance of one ohm (ω), will produce a current of one ampere. When two charges have a difference of potential the electric force that results is called electromotive force (EMF). The terms “potential”, “electromotive force” and “voltage” are used herein interchangeably. In an embodiment, the second module comprises an amplifier, and in a further embodiment, it comprises a lock-in amplifier. The difference in voltage can further be converted into impedance measures using the following equation:
R=E/I
wherein
   R is the impedance in Ohm (O);
   E is the voltage in Volt (V); and
   I is the current in Ampere (Amp).

In yet another aspect, the present invention provides an apparatus comprising a third module for measuring the impedance between the detecting and the counter electrodes. The third module may, for example, be connectable to the detecting and counter electrodes. In an embodiment, the third module is connected to the detecting and counter electrodes. As used herein, the term “impedance” is defined as a measure in ohms of the degree to which an electric circuit resists the flow of electric current when a voltage is impressed across its terminals. Impedance may also be expressed as the ratio of the voltage impressed across a pair of terminals to the current flow between those terminals. The resistance depends upon the number of electrons that are free to become part of the current and upon the difficulty that the electrons have in moving through the circuit.

In a further aspect, the present invention provides an apparatus comprising a forth module for comparing the measured impedance with a control impedance. The forth module may, for example, be connectable to the third module described above. In an embodiment, the forth module is connected to the third module described above. In an embodiment, the control impedance may be an impedance of the same sample calculated at an earlier time (e.g. T₀, after inoculation of the sample or before incubation of the sample), an impedance of a control sample substantially free of a microorganism or a reference impedance. As used herein, the term “substantially free of a microorganism” refers to a sample that does not contain a detectable amount of the microorganism that is being investigated. As such, the term “substantially free of a microorganism” includes samples free of any microorganisms as well as sample that contain microorganisms other than those that are being investigated. As used herein, the term “reference impedance” refers to an impedance value obtained in controlled experimental conditions. As such, for specific experimental conditions, a reference impedance can be predetermined and used in other situations using similar experimental conditions to evaluate the control impedance. In an embodiment, the forth module may generate normalized impedance data. As used herein, the term “normalized impedance” means the ratio of the impedance obtained for the sample at a specific point in time over the impedance obtained for the same sample at an earlier time (e.g. at T₀, right after inoculation or before incubation) or a reference impedance. The normalized impedance may further be plotted in function of time (e.g. refer to the Examples below).

In a further aspect, the present invention provides an apparatus that comprises a capture molecule. In an embodiment, the capture molecule is connectable to the detecting electrode. In an embodiment, the capture molecule is connected to the detecting electrode. In another embodiment, the capture molecule may be able to bind to a microorganism. As used herein, the term “capture molecule” is referred to as a compound that facilitates the binding of the microorganism to the detecting electrode. In an embodiment, the capture molecule is specific to a type of microorganism (e.g. a bacterium), a species of microorganism (e.g. Escherichia sp.) or even a strain of microorganism (e.g. Escherichia coli) or isolates thereof (e.g. Escherichia coli K91). In that respect, the capture molecule may confer a certain specificity to apparatus by allowing the binding of certain types, species or strains of microorganisms and/or inhibiting the binding of other types, species or strains of microorganisms. In another embodiment, the capture molecule may also be able to discriminate between two very closely related microorganisms. For example, the capture molecule can be specific (e.g. can bind to) to a microorganism that is cytotoxic and may not be able to bind to a very closely related microorganism that is not cytotoxic. As such, the capture molecule may render the apparatus able to discriminate between cytotoxic and non-cytotoxic microorganisms. In an embodiment, the capture molecule may be a nucleic acid, a polypeptide, a carbohydrate, a lipid or a combination thereof. In another embodiment, the capture molecule may be an antibody (e.g. antibody such as a polyclonal serum or a monoclonal antibody, antigen-binding fragment thereof or Fab), a phage or fragment thereof, an amino acid, a peptide, a protein or a combination thereof. In an embodiment, the capture molecule is an antibody (e.g. an antibody specific to a bacterium such as Escherichia coli). In another embodiment, the capture molecule may be a phage (e.g. a phage capable of binding or infecting a bacterium such as Escherichia coli). In a further embodiment, the capture molecule is an amino acid (e.g. alanine, asparagine, cysteine, aspartic acid, glutamic acid, phenylalanine, glycine, histidine, isoleucine, lysine, leucine, methionine, proline, glutamine, arginine, glutamine, arginine, serine, threonine, valine, tryptophan, tyrosine, glutamine, any other amino acid or any combination thereof). In another embodiment, the capture molecule is a protein such as casein. In an embodiment, the capture molecule does not alter the viability of the microorganism detected and/or does not alter the ability of the microorganism to replicate. In a further embodiment, the detecting electrode may comprise more than one type of capture molecule, thereby allowing the detection of various microorganism with the use of a single electrode.

In a further embodiment, the capture molecule and/or the detecting electrode may be adapted to modulate the affinity of the capture molecule for the detecting electrode, to modulate the affinity of the detecting electrode for the capture molecule and/or to modulate the signal (e.g. voltage and/or impedance). As such, various moieties (such as a thiol moiety) may be added to the capture molecule. Alternatively, various moieties (e.g. protein A, protein G, etc.) may also be added to the detecting electrode. Further, various moieties may be added to the detecting electrode and to the capture molecule, provided that the respective moieties may be connected to one another. For example, the detecting electrode may be coupled to streptavidin and the capture molecule may be coupled to biotin.

In yet another aspect, the present invention provides an apparatus for detecting a viable microorganism. As used herein, the term “viable” is intended to mean the capacity of a microbial cell (or microorganism) to perform its intended functions. The cellular functions may vary according to the type of cell. Cellular functions may include, for example, cellular division, cellular replication, translation, transcription, protein assembly and maturation, protein secretion, storage of compounds (e.g. proteins, lipids, etc.), responsiveness to external stimuli, migration, etc. As such, an apparatus for detecting a “viable” microorganism in a sample is an apparatus that does not irreversibly alter the microorganism's ability to perform its intended function (e.g. it does not induce cell death by apoptosis or necrosis in a majority of microorganism, nor does it cause an alteration in the functions of a majority of microorganism). In an embodiment, the apparatus may also facilitate the replication of the microorganism, provided that the microorganism is (directly or indirectly) bound to the detecting electrode. In an embodiment, the microorganism detected by the apparatus may be selected from the group consisting of a bacterium, a fungus, a mold, a spore, a virus and a prion. In an embodiment, the microorganism is a bacterium, and, in a further embodiment, the microorganism is Escherichia coli.

In an embodiment, if the microorganism to be detected requires the presence of a host cell to replicate (e.g. when the microorganism is a bacterium (such as Chlamydia sp.), a virus or a prion), the capture molecule may be such host cell. More specifically, upon infection of the host cell (e.g. binding of the microorganism to the capture molecule and/or entry of the microorganism in the host cell), the microorganism may divide and replicate into the host cell, thereby modifying the host behavior (e.g. lysis and/or detachment of the cell) and ultimately modulating the measured voltage or the measured impedance.

In an embodiment, the apparatus may be used to detect a single type of microorganism in the sample or may be adapted to detect various microorganism in the sample. In order to detect various microorganism in the sample, the apparatus may comprise more than one detecting electrode, each electrode having a single type of capture molecule and/or more than one type of capture molecule per detecting electrode.

In yet another embodiment, the apparatus may be used to detect microorganism in various samples. The samples may be a biological or a non-biological sample. The samples may be processed before their use in the apparatus (e.g. freeze, dried, semi-purified, etc.). The sample may be a solid, liquid, gaseous or a combination thereof. In an embodiment, the sample may be dissolved in a liquid media prior to its use with the apparatus. In another embodiment, a redox mediator (such as a potassium ferrocyanide/potassium ferricyanide solution) may be added to the sample prior to its used in the apparatus.

In another embodiment, the apparatus can measure the same sample at different time intervals. In yet another embodiment, the sample can be submitted to experimental conditions that enable the growth of the microorganism to be detected between measurements. For example, the samples can be incubated at a specific temperature for a specified amount of time between measurements. In an embodiment, the temperature may be about 37° C. The time intervals between the measurements may be of about 1 second to several minutes. The overall measurement time can vary from 1 hour to a few days. In another embodiment, the time intervals between the measurements may be of about 2 hour to about 4 hours.

In a further embodiment, the sample may be incubated at a specific temperature (e.g. a temperature allowing the replication of the microorganism to be detected, such as 37° C.). In another embodiment, the apparatus can be adapted to measure the voltage or the impedance of the sample at various intervals. The measurement can be discrete or made over a specified period of time (e.g. sampling time). These various measurements (or the resulting calculations) can be plotted to obtain a representative graphical representation of the behavior of the sample or used for comparison analysis.

In yet another embodiment, the apparatus may be adapted to record measurements during a specified period of time. As used herein, this specified period of time is referred to as the sampling time. The sampling time may be, for example, two minutes. The sampling time may also be adjusted to suit the detection conditions used.

In a further embodiment, the apparatus may be adapted to be used with Petri dishes, multiwell plates or any conventional receptacles used to replicate microorganisms.

In another aspect, the invention also provides methods for detecting a viable microorganism. In an embodiment, the method comprises providing a detecting electrode and a counter electrode. Various embodiments of the detecting electrode have been described above. The detecting electrode may comprise gold nanoparticles deposited thereon. Various embodiments of the gold nanoparticles have been described above. The detecting electrode may preferably also comprise a capture molecule. Such capture molecule may, for example, be able to bind to the microorganism. Various embodiments of the capture molecule have been described above. In another embodiment, the method may also comprise contacting a media-comprising sample with the detecting electrode. Such media-comprising sample may contain a viable microorganism and/or a redox mediator (such as a potassium ferrocyanide/potassium ferricyanide solution). Various embodiments of the sample have been described above. In a further embodiment, the method further comprises applying an electrical signal to the sample. Various embodiments of the electrical signal have been described above. In yet another embodiment, the method comprises measuring a difference in voltage between the detecting electrode and the counter electrode. In a further embodiment, the method comprises comparing the measured difference in potential with a control difference in potential between the detecting electrode and the counter electrode. In an embodiment, the control difference in potential may be a difference in potential between the detecting electrode and the counter electrode of the same sample calculated at an earlier time, a difference in potential between the detecting electrode and the counter electrode of a control sample substantially free of a microorganism and a reference difference in potential. As used herein, the term “reference difference in potential” refers to a difference of potential between the detecting electrode and the counter electrode obtained in controlled experimental conditions. As such, for specific experimental conditions, the reference difference in potential can be predetermined and used in other situations using similar experimental conditions to evaluate the control difference in potential. In yet a further embodiment, the method comprises calculating the impedance of the difference in potential measured by the above-described method. In still a further embodiment, the method also comprises the comparison between the measured difference in impedance and the control difference in impedance. Various embodiments of the control impedance have been described above. In yet another embodiment, the method comprises measuring a first impedance between the detecting electrode and the counter electrode. In a further embodiment, the method comprises comparing the measured first impedance with a control impedance. Various embodiments of the control impedance have been described above. In another embodiment, an increase of the first impedance with respect to the control difference in impedance is indicative of the presence of a viable microorganism. In still another embodiment, the method can be applied to the detection of a microorganism. Various embodiments of the microorganism have been described above.

In yet another aspect, the invention also provides a method for detecting a viable microorganism. In an embodiment, the method comprises providing a detecting electrode and a counter electrode. Various embodiments of the detecting and counter electrode have been described above. In another embodiment, the method also comprises contacting a media-comprising sample containing a redox mediator with the detecting electrode and the counter electrode. Various embodiments of the sample and of the redox mediator have been described above. In yet another embodiment, the method also comprises measuring a first impedance between the detecting electrode and the counter electrode. In still another embodiment, the method comprises comparing the first impedance with a control impedance. Various embodiments of the control impedance have been described above. In yet still another embodiment, an increase of the first impedance with respect to the control impedance is indicative of the presence of said viable microorganism.

In still another aspect, the invention also provides the use of a detecting electrode and a counter electrode for the detection of a microorganism in a sample. Various embodiments of the detecting electrode have been described above. The detecting electrode may preferably comprise, for example, gold nanoparticles deposited thereon. Various embodiments of the gold nanoparticles have been as described above. The detecting electrode may preferably also comprise a capture molecule. Such capture molecule may, for example, be able to bind to the microorganism. Various embodiments of the capture molecule have been described above. Further, various embodiments of the sample have been described above. In a further embodiment, the detecting and counter electrodes may be connectable to a first module for applying an electrical signal. Various embodiments of the electrical signal have been described above. In yet another embodiment, the detecting and counter electrodes may be connectable to a second module for measuring a difference in voltage between the detecting electrode and the counter electrode. Various embodiments of the second module have been described above. In a further embodiment, the detecting electrode and counter electrode may be connectable to a third module for measuring the impedance between the detecting electrode and counter electrode. In still a further embodiment, the third module may be connectable to a forth module for comparing the measured impedance with a control impedance. Various embodiments of the control impedance have been described above. In still another embodiment, the use can be applied to the detection of a microorganism. Various embodiments of the microorganism have been described above.

The present invention will be more readily understood by referring to the following examples which are given to illustrate the invention rather than to limit its scope.

### EXAMPLE I

### Preparation of the Phases

Polyclonal antibodies and biotinylated antibodies to E. coli K91 were purchased from Fitzgerald Industries International (Concord, Mass.) and Biodesign International (Saco ME). E. coli K91 was obtained from ATCC and grown in LB broth.

Phages specific to E. coli K91 (Biophage Pharma Inc, Montreal, Quebec, Canada) were produced in the range of 10¹⁰-10¹² phages/mL by an amplification procedure using E. coli K91 as the host. The multiplicity of infection (MOI) used for the initial infection was 1 phage/100 bacteria. A mixture of 10⁵ phages and 10⁷ bacteria was incubated for 15 min to optimize the initial infection before the mixture (1-2 mL) was added to 250 mL of LB broth. After 3.5 h at 37° C., the resulting culture was centrifuged at 3,500 rpm for 20 min to remove cell debris and cells; then the supernatant was filtered with a 0.22 μm syringe filter. The filtrate was purified by polyethylene glycol (PEG-8,000) precipitation. After adding 1 M NaCl to the filtered supernatant followed by 1 h of incubation, the phages were precipitated in the presence of 10% PEG after 2 h of incubation on ice. The sample was then centrifuged at 11,000 g for 20 min and the resulting pellets were brought-up in 2-3 mL of 10 mM HEPES, pH 7.4 buffered saline (150 mM NaCl). Samples were also brought-up in HEPES buffered saline containing 5 mM MgCl₂ to test for phage stability. The PEG was removed from the resuspended pellet by equilibrium dialysis (10 K molecular weight cut-off or MWCO). The sample was stable at 5° C. for weeks with a titer of 10¹²-10¹³ phages/mL, representing a typical yield ranging from 30-90%.

### EXAMPLE II

### Standard Electrodes

The multiwell ECIS biosensor system (model 100, Applied Biophysics, Troy, N.Y., USA) has the capability of simultaneous measurements up to 16 individual cultures. Both data acquisition and processing were performed using software supplied by Applied Biophysics. Each ECIS disposable electrode array consists of eight gold film electrodes (surface area: 0.5×10⁻³ cm² or 250 μm in diameter) and delineated with insulating films with a much larger common counter electrode (surface area: 0.2 cm²) located at the base of 10-mm square wells (volume ˜0.5 mL). Custom arrays with 100 μm diameter gold electrode surfaces were also obtained from Applied Biophysics.

Antibodies directed against E. coli K91 (100 μg/mL-500 μg/mL) in HEPES buffer pH 7.4 were placed (0.25 mL) in the ECIS wells overnight (at room temperature) to allow for complete binding to the gold electrodes. The wells were extensively washed to remove unbound antibody. For biotinylated antibodies (100 μg/mL-500 μg/mL) the wells were treated first for 4-6 h with 250 μg/mL avidin followed by extensive rinsing. Biotinylated antibodies were then incubated overnight followed by extensive rinsing of the wells. Similarly, phages (10⁸-10¹² phages/mL) of E. coli K91 were diluted in HEPES buffer pH 7.4 and added to the wells overnight. Experiments were conducted for a 2 h incubation period for phages and antibodies.

Bacterial cells samples were taken from the exponential growth phase (10⁸-10⁹ cells/mL) and diluted in LB culture medium to ˜10⁷ cells/mL. For monitoring detection limits, the cells were further serially diluted from 10⁶ down to 5 cells/mL.

LB culture medium (0.5 mL) was then placed in the wells and an equilibration experiment was run in the ECIS incubator for 2-3 h at 37° C. to acclimatize the system. E. coli K91 (˜10⁷ cells/mL) was then added (0.5 mL) to the pre-coated wells. The impedance measurements were compared to wells containing no phages or antibody over a period of 15-20 h at 37° C. Experiments were also conducted using 50% spent medium rather than fresh LB culture medium. The specificity of the phage was verified by performing experiments with B. thuringiensis (˜10⁶ cells/mL) obtained from the Felix d'Hérelle collection, instead of E. coli K91 in wells pre-coated with phage specific to E. coli K91.

The frequency was set at 4,000 Hz, the applied potential to the gold electrode was 1.5 V and the sampling time was 2 min. Data were displayed as either normalized resistance or impedance, however raw data were stored for the impedance, resistance and capacitance of each well. Further experiments were conducted at various frequencies, applied potentials and temperatures. A series of experiments was also performed using wells containing the 100 μm diameter gold surfaces.

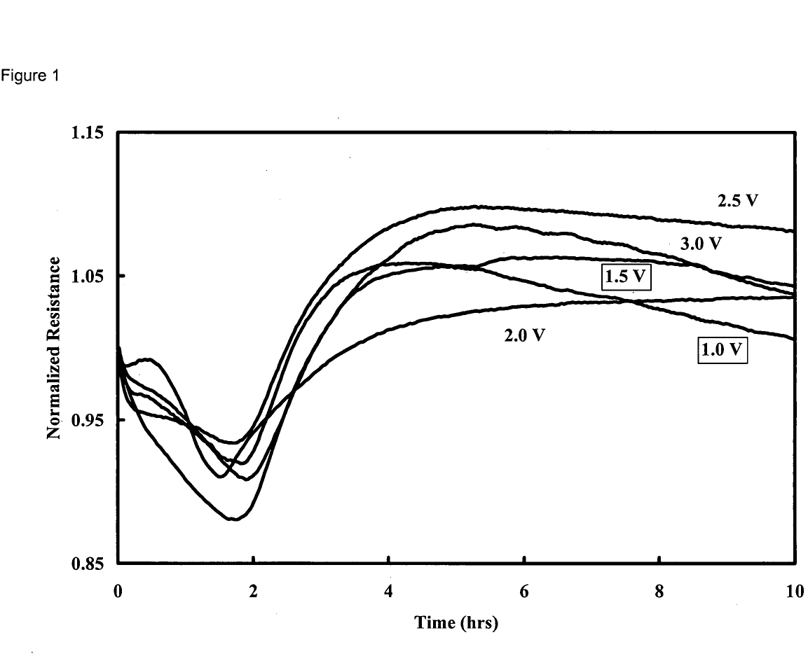

Electrodes were coated with 10¹¹ phages and incubated in the presence of 10⁷ E. coli K91. Different detecting voltage were applied and results were compared (FIG. 1). The detecting applied potential (voltage) was modified and it affected the response of the system with respect to resistance, impedance and capacitance. As the voltage was increased from 1.0 V to 3.0 V, the resistance changes were similar. The impedance or capacitance changes were optimal between 1.5 V and 2.5 V, whereas at either 1.0 V or 3.0 V, the response was much weaker. The bacterial cells are thus able to tolerate potentials as high as 2.5 V and at these higher potentials (1.5-2.5 V) all three parameters could be considered for analysis, as their sensitivities are similar. Consequently, 1.5 V was used for experiments unless otherwise stated.

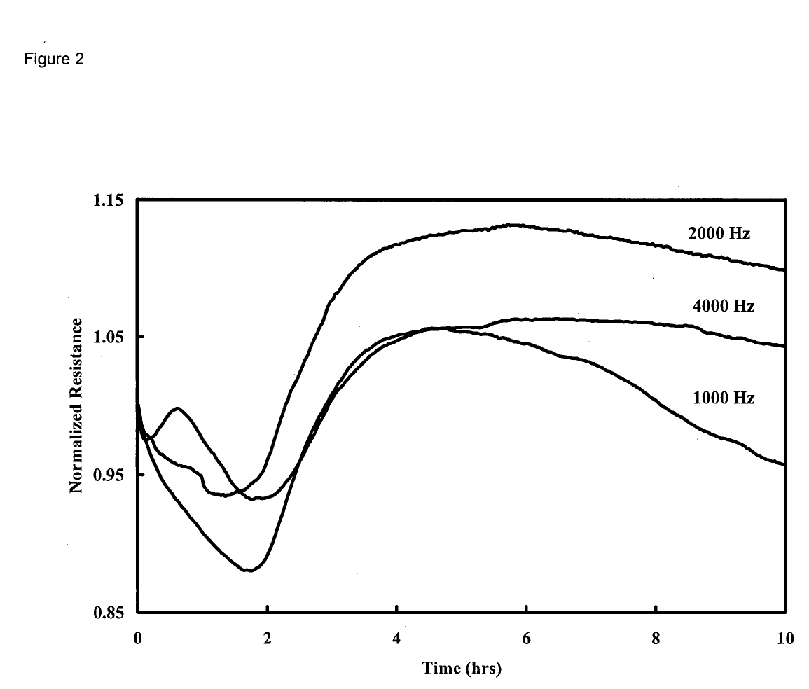

Electrodes were coated with 10¹¹ phages and incubated in the presence of 10⁷ E. coli K91. Different frequencies were applied and results were compared (FIG. 2). As the frequency was decreased from 4,000 Hz to 1,000 Hz, the resistance, impedance and capacitance changes decreased slightly, therefore frequency did not have a significant impact on the response of the system and 4,000 Hz was used for all experiments.

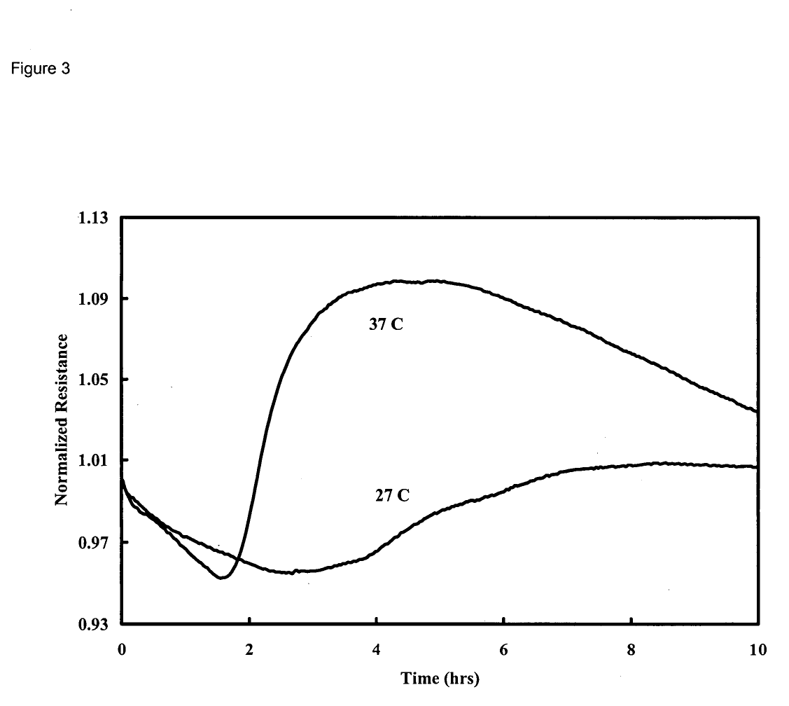

Electrodes were coated with 10¹¹ phages and incubated in the presence of 10⁷ E. coli K91. The electrodes were submitted to various incubating temperatures and results were compared (FIG. 3). As expected, the incubation temperature for the bacterial growth also affected the system response. At 27° C. the resistance response was much slower (8 h for 27° C. vs. 4 h for 37° C.) and the signal enhancement was much weaker (5% increase for 27° C. vs. 15% increase for 37° C.) than that observed at 37° C. This was expected since the E. coli growth rate will be faster at 37° C.

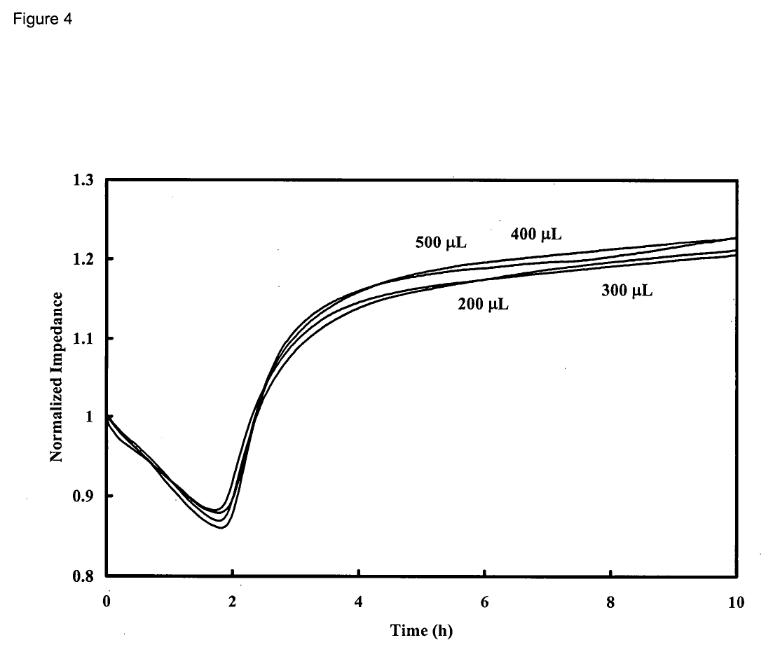

Electrodes were coated with 10¹¹ phages and incubated in the presence of 10⁷ E. coli K91. The electrodes were incubated with various inoculum volume and results were compared (FIG. 4). The inoculum volume (with 10⁷ cells/mL), monitored ranging from 200-500 μL in each well, showed no significant effect on the impedance response.

### EXAMPLE III

### Modified Electrodes

Gold electrode surfaces were modified by electrodeposition of gold nanoparticles. A 10 mM solution of hydrogen tetrachloroaurate (III) trihydrate in 0.5 M sulfuric acid was placed into a well of the ECIS chip and then both an Ag/AgCl reference and a Pt counter electrode were placed in the well. A lead was connected to the pad of the chip corresponding to the detecting electrode position of interest. The applied potential to the electrode surface was decreased from 800 mV to 200 mV in 10 s to initiate the electrodeposition of the gold nanoparticles. The gold concentration and the applied potential time were varied to monitor the effect of these parameters. Atomic force microscopy (AFM) micrographs of the resulting gold nanoparticles on the gold electrode surface were obtained using a Nanoscope IV™ (Digital Instruments, Veeco, Santa Barbara, Calif.) with a silicon tip operated in tapping mode.

The modified surface was used to monitor impedance, capacitance and resistance changes during the attachment of potential capture molecules, such as phages as mentioned above. The modified surface was also used to test the sensitivity of the system towards E. coli K91 and B. thuringiensis as described above. Data were displayed as normalized impedance (the impedance obtained before and after cell inoculation). Modified electrodes give higher impedance response compared to non-modified electrodes. In addition the modified electrodes give more reproducible results at low cell concentration.

### EXAMPLE IV

### Redox Mediator

The redox mediator couple potassium ferrocyanide/potassium ferricyanide (2.5 mM each) was mixed with LB broth and added to ECIS wells (with or without gold electrode surface modification by electrodeposition of gold nanoparticles) and an equilibration experiment was run in the ECIS incubator for 2-3 h at 37° C. to acclimatize the system. The mediator system was then used to test the sensitivity of the system towards E. coli K91. Data were displayed as normalized impedance.

### EXAMPLE V

### Results

Antibodies

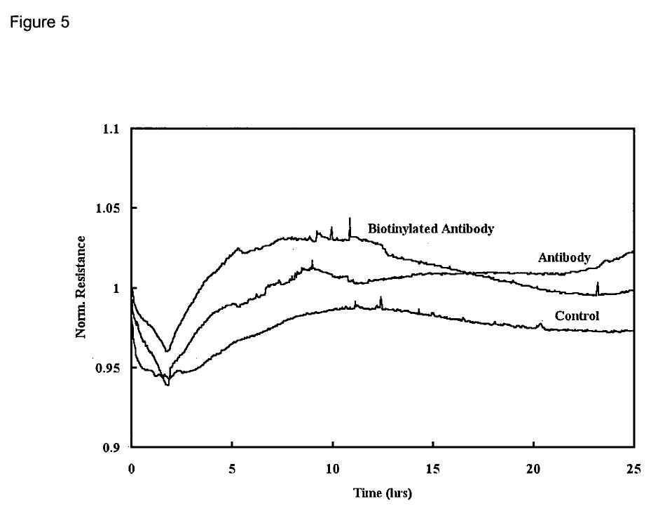

Without coating electrodes with phages or antibodies, E. coli K91 was observed to attach to the gold electrode surface very slowly, resulting in a very small increase in the resistance. As shown in FIG. 5, after an initial lag time of 2.0-2.5 h the resistance slowly increased about 2-3% (30-50 ω) over the next 10 h.

For electrodes coated with polyclonal antibodies to E. coli (FIG. 5), the impedance signal increased more rapidly as the resistance change was about 6-7% (100-120 ω) after just 6 h. Experiments with electrodes coated with biotinylated antibody (FIG. 5) resulted in an even more rapid increase in the resistance of 8-10% (120-150 ω) after 4-5 h. It should be noted that under this condition the instrument noise is in the range of a few ohms. The optimal response was achieved at antibody or biotinylated antibody concentrations of 100 μg/mL, since results at higher concentrations (250-500 μg/mL) actually provoked weaker responses.

In the case of antibodies and biotinylated antibodies to E. coli K91, the pre-incubation time was also optimum at 2 h. After inoculation with E. coli K91, the impedance response is obtained after 3 to 4 hours of incubation.

Phages

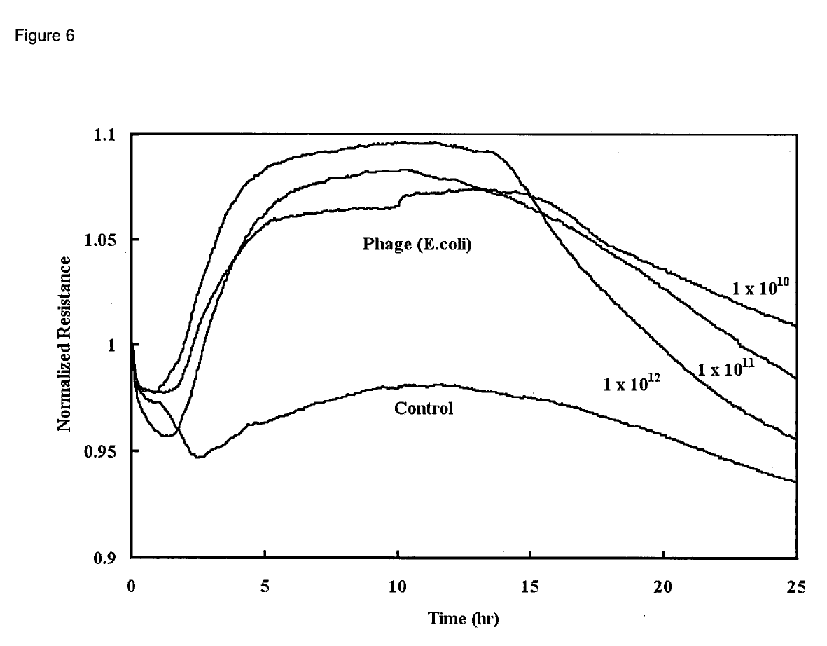

Electrodes coated with phages specific to E. coli K91 and incubated with E. coli resulted in superior impedance responses compared to antibodies (FIG. 6). The resistance increased about 12-15% and the initial lag time was shorter at about 1.5-2.0 h, while the maximum response was achieved in just 3-4 h. The maximum response was obtained at a phage concentration of about 10¹¹ phages/mL, although concentrations from 10⁹ to 10¹³ phages/mL also resulted in enhanced responses. At a concentration of 10⁸ phages/mL the response was similar to the control.

Phages stored for two weeks in a solution of HEPES buffered saline containing magnesium exhibited similar impedance profiles to phages without magnesium, indicating that magnesium was not necessary for phage stability.

It should be noted that the resistance increase was optimal at an applied potential (to the surface of the gold electrodes) of 1.5 V. At applied potentials of 1.0 V or above 2.0 V, the magnitude of the signal response was smaller and the lag time was longer. The frequency applied was optimal at 4,000 Hz, while lower frequencies (2,000 Hz) did not enhance the signal response significantly.

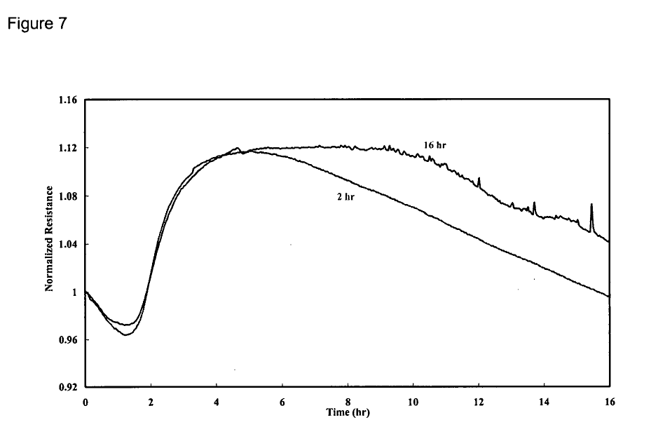

As shown in FIG. 7, the incubation time necessary for phage adhesion to the gold electrode surface was optimum at 2 h and an overnight incubation (16 h) resulted in a similar response.

Spent Medium

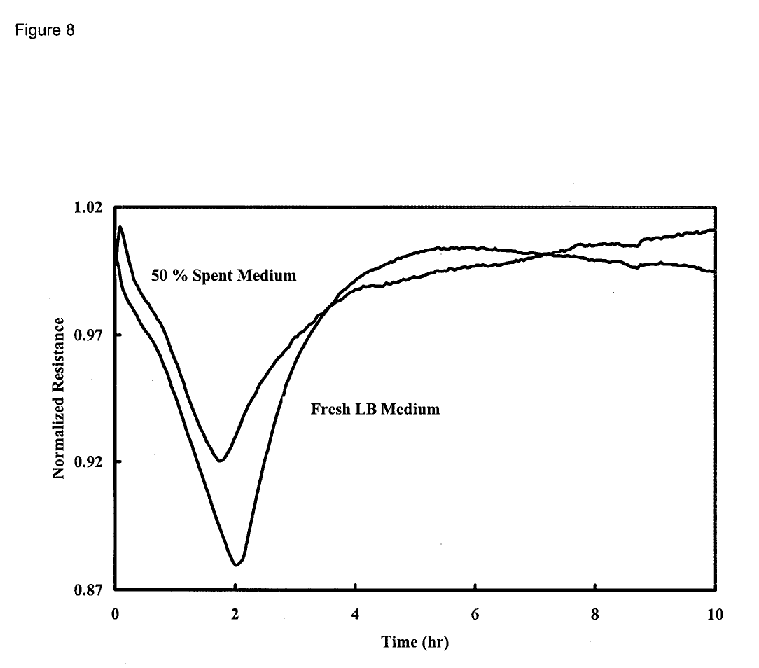

The use of 50% spent medium for E. coli K91 inoculation resulted in a weaker and slower response for wells containing phages compared to fresh LB culture medium (FIG. 8). As reported in the literature (Anal. Chem. 2001, 73, 1844-1848), the use of spent medium enhances the impedance response of insect cells, but this was not the case for bacterial cells.

Bacterial Specificity

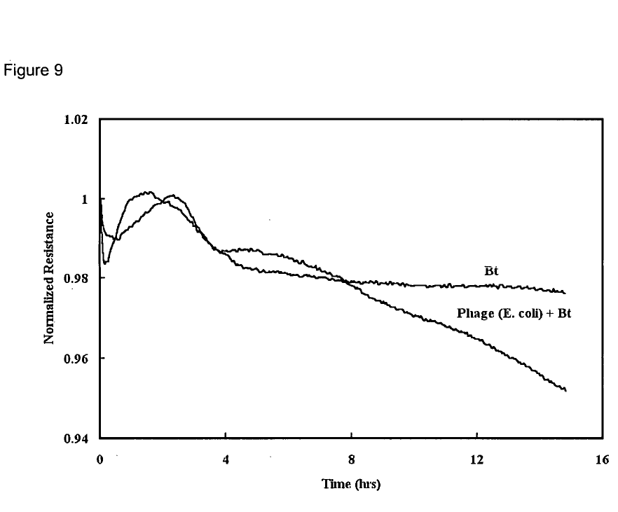

To assess the selective binding of the E. coli phage, B. thuringiensis was inoculated to electrodes pre-coated with an initial solution of 10¹¹ phages/mL of phages specific to E. coli K91 and the response was compared to wells without phages. As shown in FIG. 9, the pattern of the weak resistance response with time was similar for B. thuringiensis grown in wells with or without phages specific to E. coli K91, unlike the equivalent responses with E. coli K91. This observation confirms the specificity and selectivity of the selected phages for E. coli K91.

Bacterial Concentration

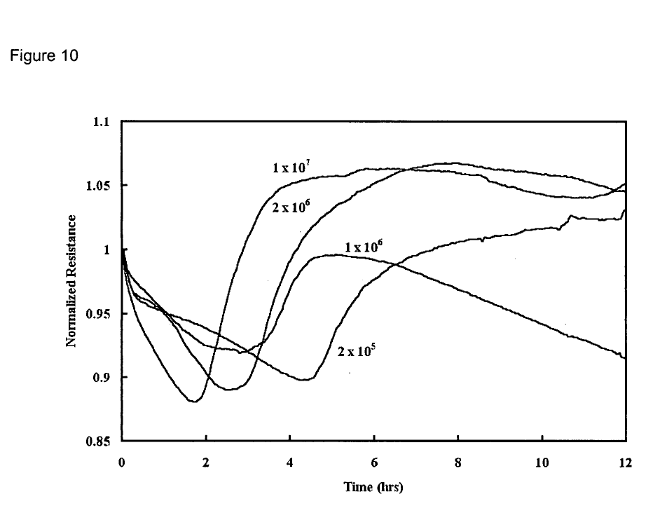

Experiments were performed using electrodes pre-incubated with 10¹¹ phages/mL to monitor the sensitivity of the response at different E. coli K91 cell concentrations. When E. coli K91 were used in the range of 10⁵-10⁷ cells/mL, the resistance response diminished as the cell concentration decreased (FIG. 10). In addition, the lag time before the resistance change was observed increased as the cell concentration decreased (FIG. 10).

Electrode Surface

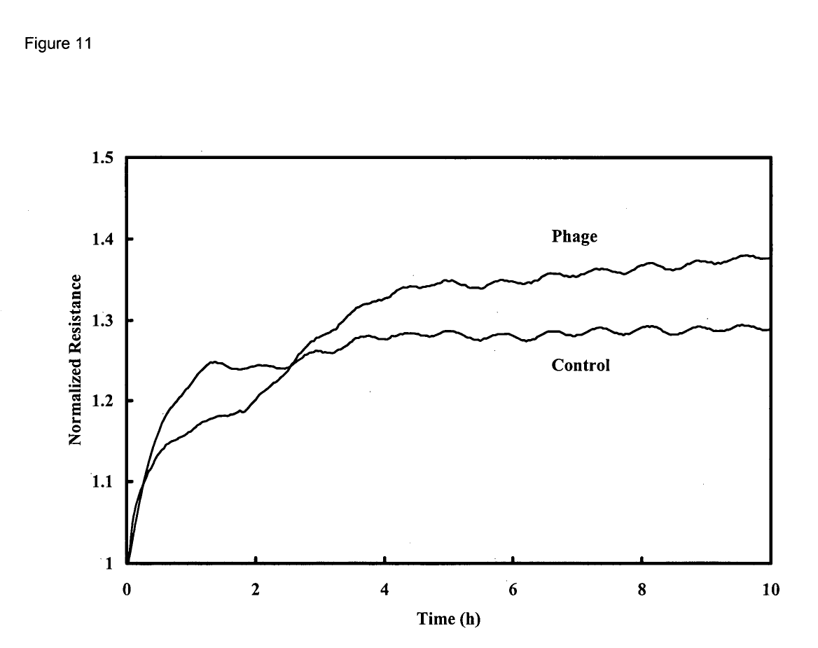

Decreasing the electrode diameter from 250 μm to 100 μm did not significantly change the resistance response (FIG. 11). The change in resistance was about 500-700 ω compared to 150 ω at 250 μm diameter gold electrodes. However, due to the smaller surface area the initial resistance values for the 100 μm gold electrodes were 4,000-6,000 ω rather than 1,500-1,800 ω observed as initial values for the 250 μm gold electrode surfaces. Therefore the normalized response was the same and a similar pattern was observed for impedance and capacitance. It should be noted that the response signals were noisier when using the smaller diameter gold electrodes.

Electrode Modification

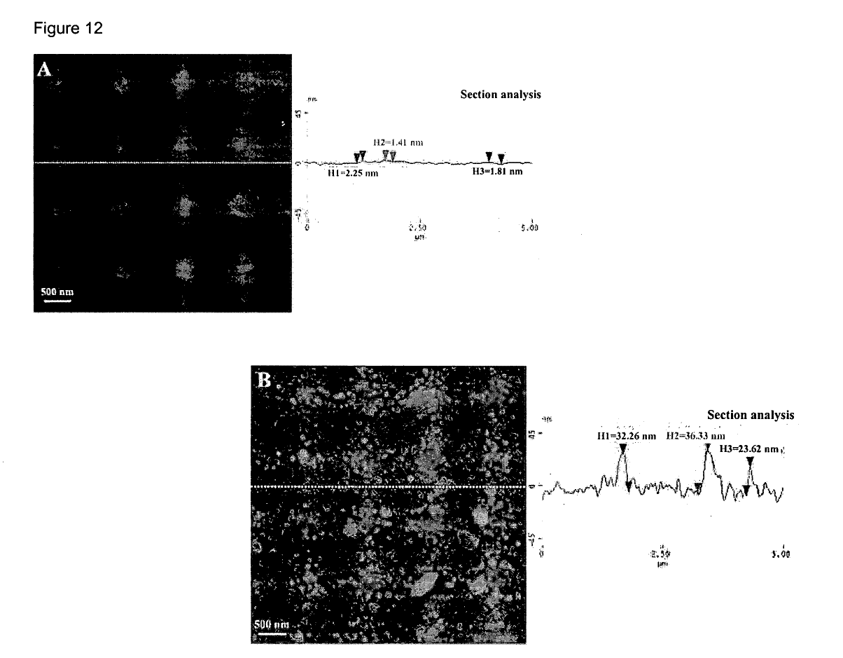

Modifying the gold electrode surface (250 μm) by electrodeposition (10 s) of gold nanoparticles from the gold salt solution (10 mM) resulted in dramatic changes in the impedance and capacitance whereas the resistance remained unchanged. Uncoated modified electrodes were incubated with an initial E. coli K91 concentration of 10⁷ cells/mL. Upon electrodeposition of the gold nanoparticles, the impedance dropped from 11,000-14,000 ω to only 2,000-4,000 ω, while the capacitance increased from 3-4 nF to 10-25 nF. The resistance remained at between 1,500-1,800 ω. The size of the gold nanoparticles was in the range of 15-40 nm as determined by atomic force image (FIG. 12B—5×5 μm) with a mean roughness of 4.12 nm. FIG. 12A shows the smooth unmodified starting gold surface with a mean roughness of 0.57 nm.

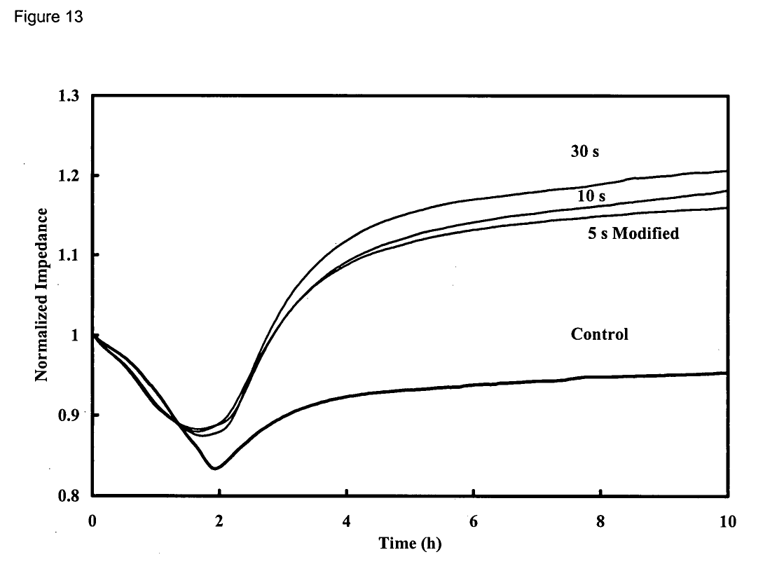

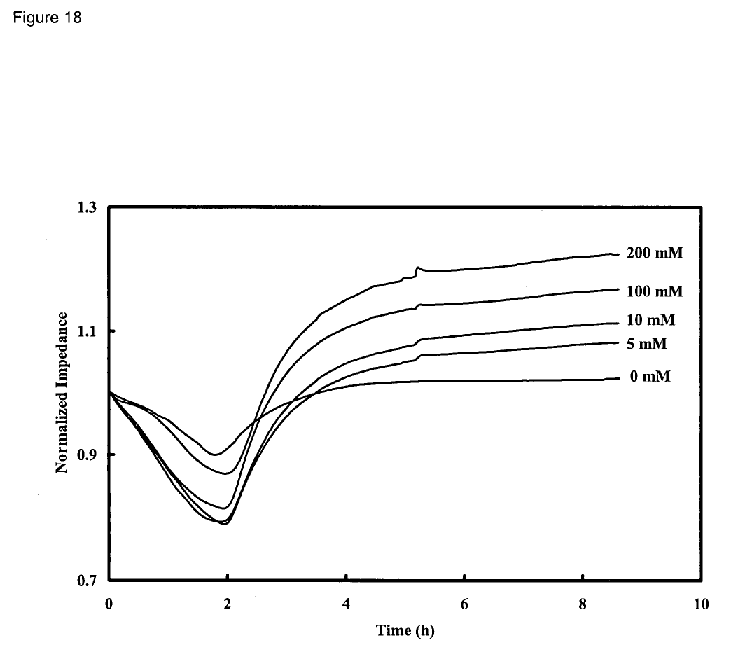

Increasing the deposition time from 5 to 30 s did not significantly affect the response for either impedance or capacitance, although as indicated earlier, the size of the gold nanoparticles on the surface became larger (FIG. 13). In the case of the resistance signal, the signal actually became weaker as the applied potential time was increased from 5 to 20 s. Varying the gold concentration from 5 to 200 mM did not significantly affect the response in either impedance or capacitance (FIG. 18). The resistance signal was weaker as the gold concentration was increased from 5 to 200 mM The response at 0 mM gold solution (potential was applied to the well containing sulfuric acid) was very similar to the results for the unmodified surface implying that the enhanced response was due to the presence of the gold nanoparticles and not simply the electrode surface treatment with the acid. Therefore, a deposition time of 10 s and a gold salt concentration of 10 mM were used to modify the gold electrode surface with gold nanoparticles. The time of applied potential was decreased to 5 s or increased to 30 s. Increasing the time of the applied potential from 10 s to 30 s increased the size of the gold nanoparticles to 200-300 nm.

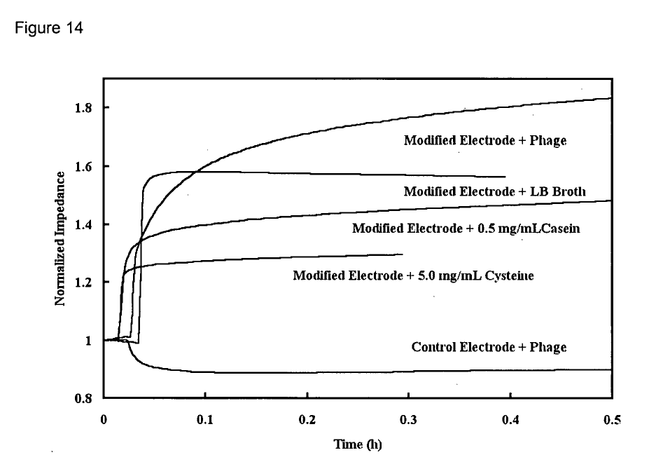

Modified electrodes were then coated with phages (FIG. 14). After the attachment of the phages, again significant changes were noted in the capacitance and impedance while the resistance remained around 1,600-2,000 ω. The impedance increased to 5,000-7,000 ω while the capacitance decreased to 6-8 nF. Therefore, the attachment of phages to the modified gold surface can be monitored by either impedance or capacitance but not resistance to determine the optimal contact time. As observed the change was very rapid (5-10 min) and a plateau was achieved after about 30-60 min. This was not feasible with the unmodified gold surface as the changes were negligible for all three measured parameters. As also shown in FIG. 14, amino acids such as cysteine, proteins such as casein (0.5 mg/mL) or the LB broth (containing 17 mg/mL of digested casein) showed similar adhesion patterns to phages.

As observed in FIG. 14, the impedance response for various capture molecules increases up to 2 fold (as in the case of phages) using the modified gold electrode surface compared to the unmodified surface, indicating a binding event for the capture molecule.

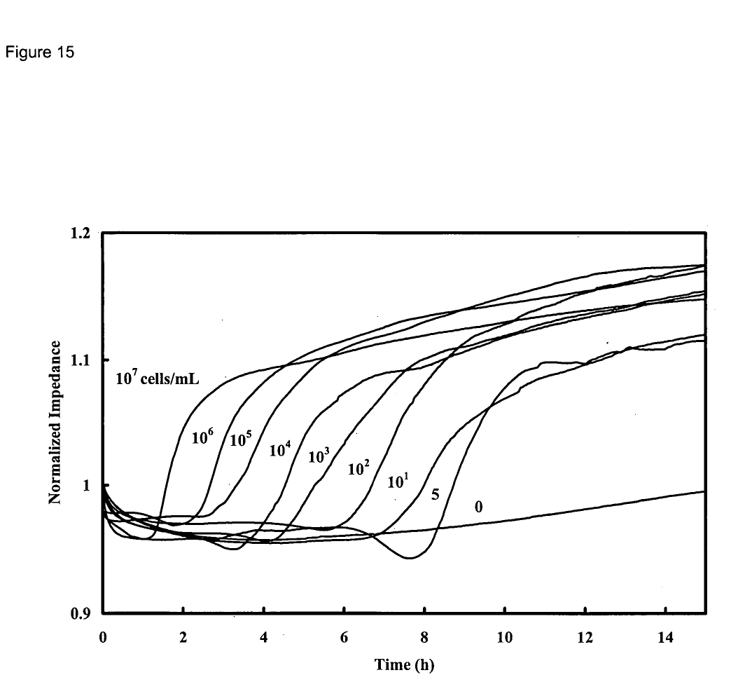

Since the gold nanoparticles on the modified surface carry a negative charge, the bacterial cells might be able to adhere directly to the modified surface, through their amino groups. Experiments were performed to monitor the sensitivity of the impedance response at different E. coli K91 cell concentrations and the detection limit was determined to be 5 cells/mL with a lag time of about 10 h. When E. coli K91 was used in the range of 10⁷ down to 5 cells/mL, the total impedance change was similar, however as the cell concentration decreased the lag time before the impedance change increased from 2 h to 10 h as expected (FIG. 15).

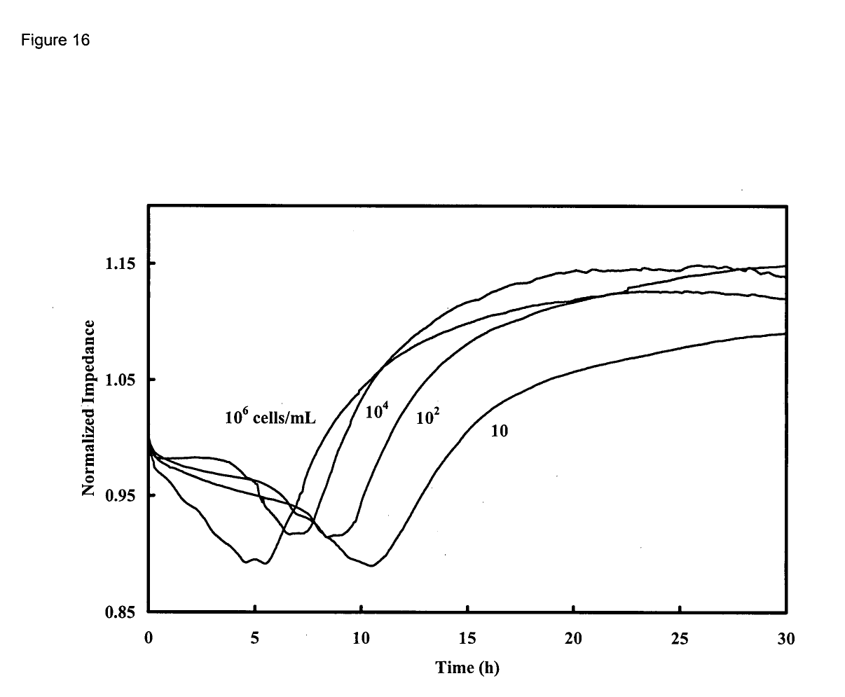

Using the modified gold electrode surface, experiments were also performed to monitor the sensitivity of the impedance response at different B. thuringiensis cell concentrations. When B. thuringiensis cells were used in the range of 10⁶ to 10 cells/mL, the total impedance change was similar, however as the cell concentration decreased the lag time before the impedance change was observed increased. Although the lag time was similar for both E. coli and B. thuringiensis at similar cell concentrations the rate of the impedance increase was faster in the case of E. coli (FIG. 16).

Modification of the small gold electrode surface (100 μm) with gold nanoparticles was also performed. Similarly to the larger electrode surface, a decrease in the impedance was observed, in this case from 45,000 ω to 15,000-25,000 ω. The capacitance increased from 0.9 nF to 2-3 nF during the same time. Once again the addition of phage to the modified surface could be monitored as an impedance increase. Impedance values increased to about 25,000-35,000 ω while the capacitance decreased to about 1.3-1.7 nF. The modified electrode surface was compared to the unmodified surface with respect to the response for E. coli K91 and there was an enhancement with the modified surface, as was the case with the larger electrode surface. However, the magnitude of the response increase was smaller compared to the larger electrode surface, and similar results were observed for capacitance and resistance changes. Using the modified gold electrode surface, experiments were then performed to monitor the sensitivity of the impedance response at different E. coli K91 cell concentrations and the results were very similar to those observed with the larger gold electrode surface area, although the amplitude of the normalized impedance response was lower.

Redox Mediator

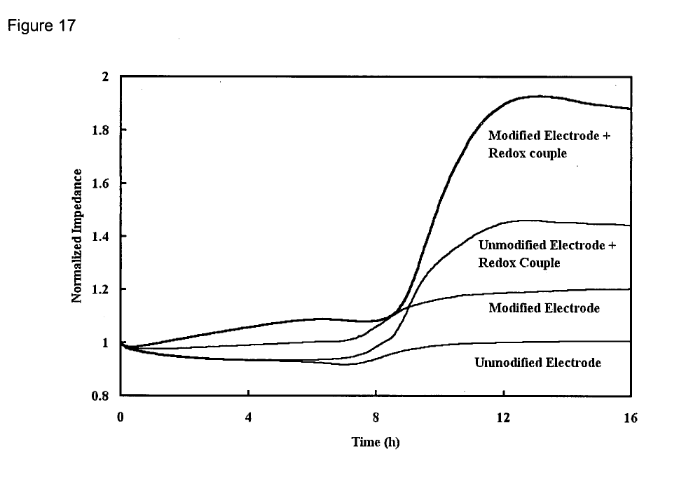

Uncoated electrodes incubated with an initial concentration of 10² E. coli cells/mL were then incubated with a redox mediator couple. Addition of the redox mediator couple potassium ferrocyanide/potassium ferricyanide to the LB growth medium in the wells resulted in a significant increase of 4-5 fold in the impedance response (FIG. 17) compared to the unmodified electrode. The concentration of the mediator couple (equal amounts of each) required to give the maximum enhancement was 1 mM, as lower concentrations (0.25 mM) gave similar responses to the control (0 mM). At very high concentrations of the mediator (20 mM) the lag time was longer for a cell concentration of 10⁶ cells/mL.

At low cell concentration (5-100 cells/mL) 2.5 mM mediator was sufficient to obtain a strong response signal, however at 5 mM the lag time was significantly increased. As a result 2.5 mM of the mediator couple was used for all experiments. The impedance signal amplitude was similar for all cell concentrations from 5×10⁷ cells/mL, while the lag time increased from 2 h to 10 h as the concentration decreased.

Addition of the redox mediator couple (2.5 mM) to the LB growth medium in the wells resulted in a significant increase of 4-5 fold in the impedance response (FIG. 17) compared to the modified electrode. The results with respect to mediator concentration and detection limit (5 cells/mL) were similar to those reported for the unmodified surface.

Once the cell concentration was below 5 cells/mL the results were governed by the statistical probability of obtaining at least 1 cell in the inoculation volume (0.5 mL). For example at 5 cell/mL, experimental repeats using 12 wells always gave a response. However, for 6 repeats at 2.0, 1.0, 0.5, 0.1 and 0 cells/mL the number of positive results was 3, 2, 1, 0, and 0, respectively. Pre-incubation of samples containing low cell concentration resulted in signals for all 8 repeated tests. For example, at 0.5 cells/mL a pre-amplification time of 3 h was needed (2 h resulted in only 6 out of 8 positive results), whereas at 0.1 cells/mL a 6 h pre-amplification was sufficient. Therefore it was easy to detect 1 cell in a 10 mL sample using a short pre-incubation step.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. An apparatus for detecting a viable microorganism in a sample, said apparatus comprising:
a detecting electrode comprising gold nanoparticles deposited thereon;
a counter electrode; and
a capture molecule, said capture molecule being connectable to said detecting electrode and said capture molecule being able to bind to said microorganism.

2. The apparatus of claim 1, wherein said detecting electrode is a gold electrode.

3. The apparatus of claim 1, wherein the average size of said gold nanoparticles is from about 15 nm to about 300 nm.

4. The apparatus of claim 1, wherein the diameter of said detecting electrode is of about 100 μm to about 250 μm.

5. The apparatus of claim 1, said apparatus further comprising a first module for applying an electrical signal to said sample, said first module being connectable to said detecting electrode and said counter electrode.

6. The apparatus of claim 5, wherein said electrical signal has an alternating current from about 1 μA to about 3 μA.

7. The apparatus of claim 5, wherein said electrical signal has a potential of about 1.0 V to about 3.0 V.

8. The apparatus of claim 5, wherein said electrical signal has a potential of about 1.5 V.

9. The apparatus of claim 5, said apparatus further comprising a second module for measuring a difference in voltage between said detecting electrode and said counter electrode, said second module being connectable to said detecting electrode and said counter electrode.

10. The apparatus of claim 9, wherein said second module comprises an amplifier.

11. The apparatus of claim 5, said apparatus further comprising a third module for measuring a first impedance between said detecting electrode and said counter electrode, said third module being connectable to said detecting electrode and said counter electrode.

12. The apparatus of claim 11, said apparatus further comprising a forth module for comparing said first impedance with a control impedance, said forth module being connectable to said third module.

13. The apparatus of claim 12, wherein said control impedance is selected from the group consisting of an impedance of said sample measured at an earlier time, an impedance of a control sample substantially free of microorganism and a reference impedance.

14. The apparatus of claim 1, wherein said capture molecule is selected from the group consisting of an antibody, a phage, an amino acid and a protein.

15. The apparatus of claim 14, wherein said antibody is directed against Escherichia coli.

16. The apparatus of claim 14, wherein said phage is capable of binding to Escherichia coli.

17. The apparatus of claim 1, wherein said microorganism is selected from the group consisting of a bacterium, a fungus, a mold, a spore, a virus and a prion.

18. The apparatus of claim 1, wherein said microorganism is a bacterium.

19. The apparatus of claim 19, wherein said bacterium is Escherichia coli.

20. A method for detecting a viable microorganism, said method comprising:
a) providing a detecting electrode and a counter electrode, said detecting electrode comprising (i) gold nanoparticles deposited thereon and (ii) a capture molecule, said capture molecule being able to bind to said microorganism;
b) contacting a media-comprising sample with said detecting electrode and said counter electrode;
c) measuring a first impedance between said detecting electrode and said counter electrode; and
d) comparing said first impedance with a control impedance, said control impedance being selected from the group consisting of an impedance between said detecting electrode and said counter electrode of said sample at an earlier time, an impedance between said detecting electrode and said counter electrode in a sample substantially free of microorganism and a reference impedance;
wherein an increase of said first impedance with respect to said control impedance is indicative of the presence of said viable microorganism.

21. The method of claim 20, wherein said detecting electrode is a gold electrode.

22. The method of claim 20, wherein the average size of said gold nanoparticles is from about 15 nm to about 300 nm.

23. The method of claim 20, wherein the diameter of said detecting electrode is of about 100 μm to about 250 μm.

24. The method of claim 20, said method comprising applying an electrical signal to said media-comprising sample.

25. The method of claim 24, wherein said electrical signal has an alternating current from about 1 μA to about 3 μA.

26. The method of claim 24, wherein said electrical signal has a potential of about 1.0 V to about 3.0 V.

27. The method of claim 24, wherein said electrical signal has a potential of about 1.5 V.

28. The method of claim 20, wherein said capture molecule is selected from the group consisting of an antibody, a phage, an amino acid and a protein.

29. The method of claim 28, wherein said antibody is directed against Escherichia coli.

30. The method of claim 28, wherein said phage is capable of binding to Escherichia coli.

31. The method of claim 20, wherein said microorganism is selected from the group consisting of a bacterium, a fungus, a mold, a spore, a virus and a prion.

32. The method of claim 20, wherein said microorganism is a bacterium.

33. The method of claim 32, wherein said bacterium is Escherichia coli.

34. The method of claim 20, said method further comprising adding a redox mediator to said media-comprising sample.

35. The method of claim 34, wherein said redeox mediator is a potassium ferrocyanide/potassium ferricyanide solution.

36. A method for detecting a viable microorganism, said method comprising:
a) providing a detecting electrode and a counter electrode;
b) contacting a media-comprising sample containing a redox mediator with said detecting electrode and said counter electrode;
c) measuring a first impedance between said detecting electrode and said counter electrode;
d) comparing said first impedance with a control difference in impedance, said control impedance being selected from the group consisting of an impedance between said detecting electrode and said counter electrode of said sample at an earlier time, an impedance between said detecting electrode and said counter electrode in a sample substantially free of microorganism and a reference difference in impedance; and
wherein an increase of said first impedance with respect to said control impedance is indicative of the presence of said viable microorganism.
